(19) United States
(12) Patent Application Publication
(10) Pub. No.: US 2007/0238930 A1
(43) Pub. Date: Oct. 11, 2007

(54) ENDOSCOPE TIPS, SCANNED BEAM ENDOSCOPES USING SAME, AND METHODS OF USE

(76) Inventors: Christopher A. Wiklof, Everett, WA (US); John R. Lewis, Bellevue, WA (US); Selso Luanava, Woodinville, WA (US)

Correspondence Address: Michael G. Pate, Esq.;DORSEY & WHITNEY LLP, Suite 3400, 1420 Fifth Avenue, Seattle, WA, 98101, US

(21) Appl. No.: 11/679,115

(22) Filed: Feb. 26, 2007

Related U.S. Application Data
(60) Provisional application No. 60/777,695, filed on Feb. 27, 2006.

(51) Int. Cl. A61B 1/07

(52) U.S. Cl. 600/160

(57) ABSTRACT

Apparatuses and methods for scanned beam endoscopes, endoscope tips, and scanned beam imagers are disclosed. In one aspect, a scanned beam endoscope includes at least one light detection element that collects light reflected from a FOV through one or more openings in the scanner of the endoscope. In another aspect, the illumination optical fiber may be positioned so that its output end is laterally positioned in relation to the scanner. In yet another aspect, the scanner is oriented to provide a non-axial FOV.

### CROSS-REFERENCE TO RELATED APPLICATION

This application is based on provisional application No. 60/777,695, filed Feb. 27, 2006.

### TECHNICAL FIELD

This invention relates to scanned beam systems and, more particularly, to scanned beam endoscopes.

### BACKGROUND

Video endoscopes have been in general use since the 1980s for viewing the inside of the human body. Endoscopes are typically flexible or rigid devices that have an endoscope tip including an imaging unit, such as a digital camera or a scanned beam imager, configured for collecting light and converting the light to an electronic signal. The electronic signal is sent up a flexible tube to a console for display and viewing by a medical professional such as a doctor or nurse.

Scanned beam endoscopes are a fairly recent innovation, and an example of a scanned beam endoscope is disclosed in U.S. patent application Ser. No. 10/873,540 (“'540 application”) entitled SCANNING ENDOSCOPE, hereby incorporated by reference and commonly assigned herewith. FIGS. 1 through 3 show a scanned beam endoscope disclosed in '540 application. As shown in FIG. 1, the scanned beam endoscope 100 includes a control module 102, monitor 104, and optional pump 106, all of which may be mounted on a cart 108, and collectively referred to as console 110. The console 110 communicates with a handpiece 112 through an external cable 114, which is connected to the console 110 via connector 116. The handpiece 112 is operably coupled to the pump 106 and an endoscope tip 120. The handpiece 112 controls the pump 106 in order to selectively pump irrigation fluid through a hose 126 and out of an opening of the endoscope tip 120 in order to lubricate a body cavity that the endoscope tip 120 is disposed within. The endoscope tip 120 includes a distal tip 118 having a scanning module configured to scan a beam across a field-of-view (FOV).

The endoscope tip 120 and distal tip 118 thereof are configured for insertion into a body cavity for imaging internal surfaces thereof. In operation, responsive to user input via the handpiece 1112, the scanning module of the distal tip 118 scans a beam of light over a FOV, collects the reflected light from the interior of the body cavity, and sends a signal representative of an image of the internal surfaces to the console 110 for viewing and use by the medical professional.

FIGS. 2 and 3 depict the distal tip 118 and a scanning module 128 of the distal tip 118, respectively, according to the prior art. Referring to FIG. 2, the distal tip 118 includes a housing 130 that encloses and carries the scanning module 128, a plurality of detection optical fibers 132, and an end cap 131 affixed to the end of the housing 130. The detection optical fibers 132 are disposed peripherally about the scanning module 128 within the housing 130. Referring to FIG. 3, the scanning module 128 has a housing 134 that encloses and supports a micro-electro-mechanical (MEMS) scanner 136 and associated components, an illumination optical fiber 138 affixed to the housing 134 by a ferrule 142, and a beam shaping optical element 140. A dome 133 is affixed to the end of the housing 130 and may be hermetically sealed thereto in order to protect the sensitive components of the scanning module 128.

In operation, the distal tip 118 is inserted into a body cavity. The illumination optical fiber 138 outputs a beam 144 that is shaped by the beam shaping optical element 140 to form a shaped beam 146 having a selected beam shape. The shaped beam 146 is transmitted through an aperture in the center of the MEMS scanner 136, reflected off a first reflecting surface 148 of the interior of the dome to the front of the scanner 136, and then reflected off of the scanner 136 as a scanned beam 150 through the dome 133. The scanned beam 150 is scanned across a FOV and reflected off of the interior of a body cavity. At least a portion of the reflected light from the FOV (e.g., specular reflected light and diffuse reflected light also referred to as scattered light) is collected by the detection optical fibers 132. Accordingly, the reflected light collected by the detection optical fibers 132 may be converted to an electrical signal using optical-electrical converters, such as photodiodes, and the signal representative of an image may be sent to the console 110 for viewing on the monitor 104.

While the scanned beam endoscope 100 is an effective endoscope, the distal tip 118 has a diameter that is typically larger than desired. It may be desirable to reduce the overall bulkiness and size of the distal tip 118 so that the size of an incision made for insertion of the distal tip 118 can be reduced. Reducing the size of the distal tip 118 may also be desirable to reduce patient discomfort when the endoscope is inserted into a preexisting opening in the body. Also, in some applications, it may be desirable to selectively position the illumination optical fiber 138 and/or the detection optical fibers 132 within the scanning module 128 to improve the performance characteristics of some aspects of the distal tip 118.

### SUMMARY

Scanned beam endoscopes, endoscope tips, scanned beam imagers, and methods of use are disclosed. In one aspect, a scanned beam endoscope includes a light source and an endoscope tip. The endoscope tip includes an illumination optical fiber having an output end and an input end coupled to the light source. The endoscope tip further includes a scanner positioned to receive a beam output from the output end of the illumination optical fiber and operable to scan the beam across a FOV. The scanner includes a plurality of openings extending therethrough, and the openings may be defined by the structure of the scanner such as the openings between a scan plate and gimbal and the gimbal and frame of the scanner. One or more light detection elements may be positioned to receive light reflected from the FOV through at least one of the openings in the scanner.

In another aspect, a method of collecting light reflected from a FOV is disclosed. The method includes scanning a beam across a FOV using a scanner. The method further includes transmitting at least a portion of light reflected from the FOV through at least one opening in the scanner for collection with at least one light detection element.

In another aspect, a scanned beam endoscope includes a light source and an endoscope tip. The endoscope tip includes an illumination optical fiber having an output end and an input end coupled to the light source. The endoscope tip further includes a scanner positioned to receive a beam output from the output end of the illumination optical fiber and operable to scan the beam across a FOV. The output end of the illumination optical fiber may be laterally positioned in relation to the scanner. One or more light detection elements may be positioned to receive light reflected from the FOV.

In another aspect, a method of scanning light across a FOV is disclosed. The method includes transmitting a beam from a location lateral in relation to a scanner and redirecting the beam to the scanner. The method further includes scanning the redirected beam across the FOV.

In another aspect, a scanned beam endoscope, includes a light source operable to provide light and an endoscope tip. The endoscope tip includes an optical fiber having an output end and an input end coupled to the light source and a scanner positioned to receive a beam output from the output end of the optical fiber and operable to scan the beam across a FOV. A central normal axis of the scanner is oriented at a non-zero angle relative to a longitudinal axis of the endoscope tip. A converter is provided that is operable to covert optical signals characteristic of light reflected from the FOV to electrical signals. The scanned beam endoscope further includes a display coupled to receive the electrical signals from the converter, the display being operable to show an image characteristic of the FOV.

In yet another aspect, a method of scanning a beam across a field of view (FOV) from an endoscope tip includes scanning the beam across the FOV using a scanner. A central axis of the FOV is oriented at a non-zero angle relative to a longitudinal axis of the endoscope tip.

The teachings disclosed herein are also applicable to scanned beam imagers and bar code scanners.

### BRIEF DESCRIPTION OF THE DRAWINGS

FIG. 1 is schematic drawing of a scanned beam endoscope according to the prior art.

FIG. 2 is a schematic partial isometric view of a distal tip shown in FIG. 1 according to the prior art.

FIG. 3 is a schematic partial side cross-sectional view of the scanning module of FIG. 2 according to the prior art.

FIG. 4 is a schematic isometric view of a distal tip of an endoscope tip having detection optical fibers that collect light reflected from a FOV through openings in a scanner according to one embodiment.

FIG. 5 is a schematic partial side cross-sectional view of the distal tip of FIG. 4.

FIG. 6 is a schematic front cross-sectional view of the distal tip of FIGS. 4 and 5.

FIG. 7 is a schematic partial side cross-sectional view of a distal tip of an endoscope tip in which photodiodes are positioned to receive light reflected from the FOV through openings in the scanner according to another embodiment.

FIG. 8 is a schematic front cross-sectional view of the distal tip of FIG. 7.

FIG. 9 is a schematic partial side cross-sectional view of a distal tip of an endoscope tip in which the illumination optical fiber is positioned to emit a beam from the side of the scanner according to another embodiment.

FIG. 10 is a schematic side cross-sectional view of a distal tip of an endoscope tip in which the scanner is positioned so that a central normal axis of the scanner is oriented at a non-zero angle relative to a longitudinal axis of the endoscope tip according to yet another embodiment.

FIG. 11 is schematic drawing of a scanned beam endoscope that may utilize any of the distal tips disclosed herein according to one embodiment.

FIG. 12 is a block diagram illustrating the relationship between the various components of the scanned beam endoscope of FIG. 11 according to one embodiment.

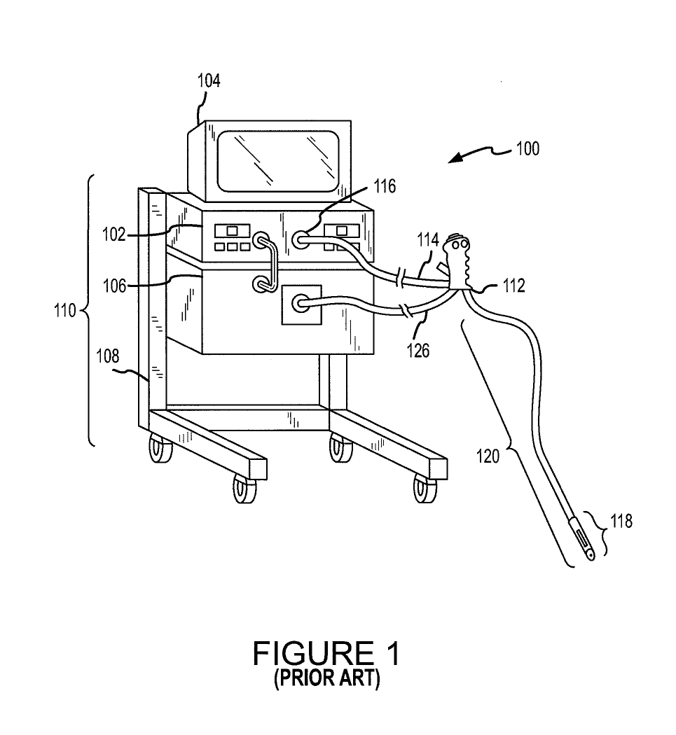

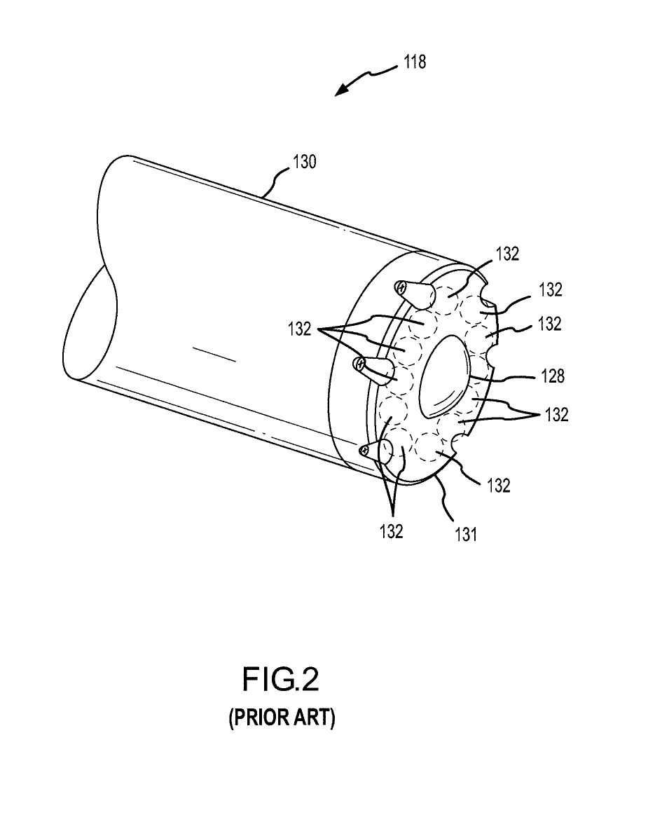

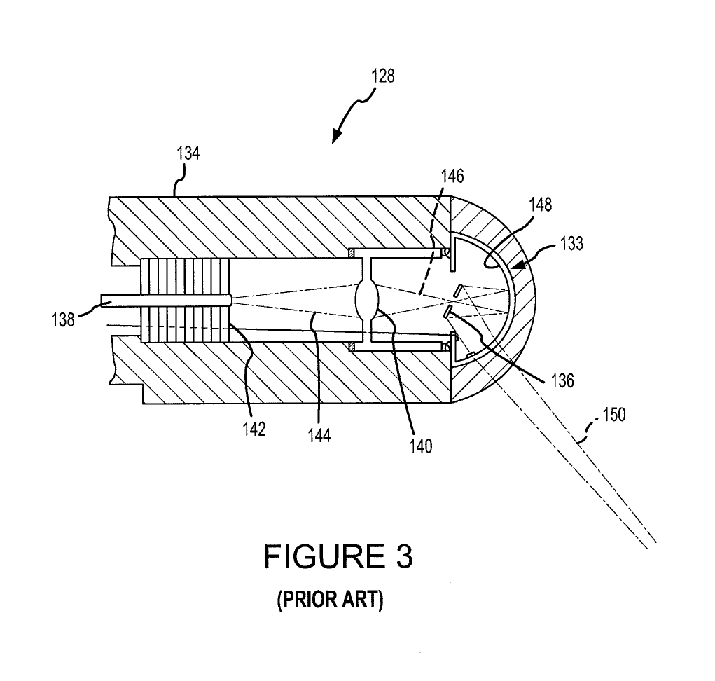

### DETAILED DESCRIPTION OF THE EMBODIMENTS

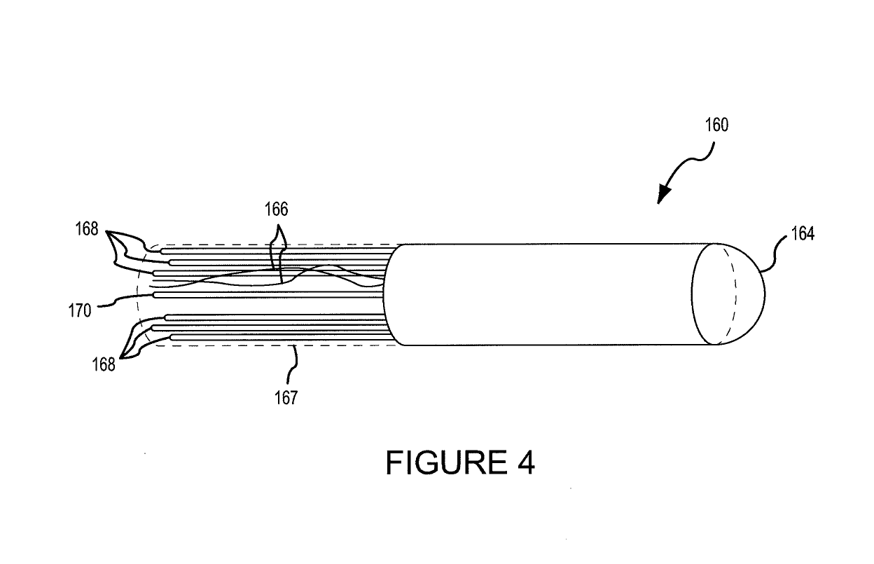

Apparatuses and methods for scanned beam endoscopes, endoscope tips, and scanned beam imagers are disclosed. Many specific details of certain embodiments are set forth in the following description and in FIGS. 4 through 12 in order to provide a thorough understanding of such embodiments. One skilled in the art, however, will understand that there may be additional embodiments, or that the disclosed embodiments may be practiced without several of the details in the following description.

FIGS. 4 through 6 show one embodiment of an distal tip 160 of an endoscope tip for use in a scanned beam endoscope that includes at least one detection optical fiber positioned to receive, through at least one opening in the scanner, light reflected from an internal body surface. FIG. 4 shows a schematic isometric view of the distal tip 160 that may form part of or all of an endoscope tip. The distal tip 160 may be attached to or contained within the distal end of a hollow body or tube 167 of an endoscope tip that encloses the electrical and optical components thereof, such as wires 166, detection optical fibers 168, and an illumination optical fiber 170. The hollow body 167 may be rigid or flexible depending upon the particular endoscope application.

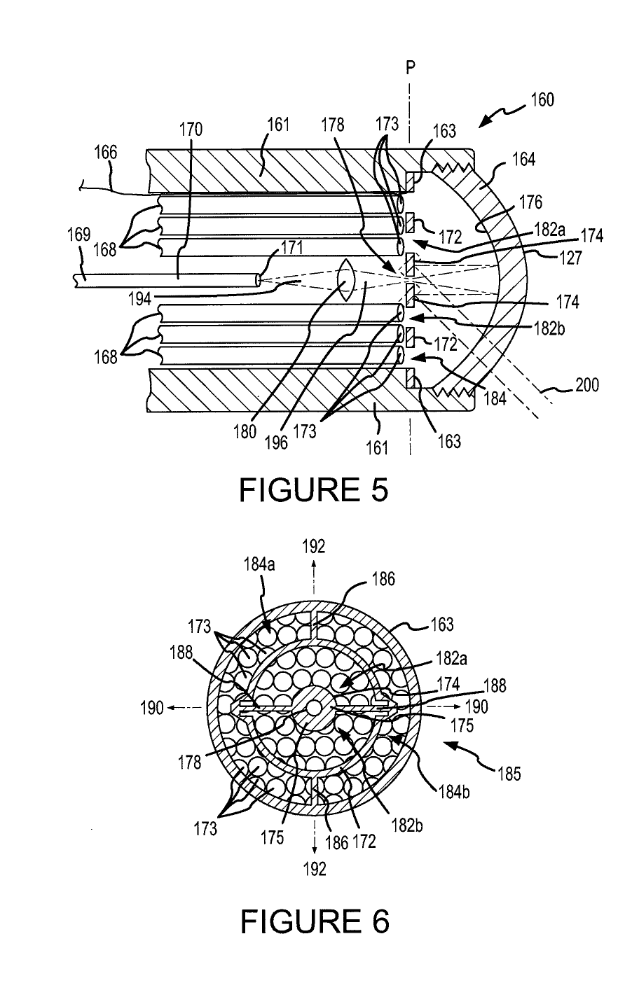

Turning now to FIGS. 5 and 6, which show the distal tip 160 of FIG. 4 in more detail as schematic partial side and front cross-sectional views, respectively. The distal tip 160 includes a housing 161 that encloses a plurality of the detection optical fibers 168, an illumination optical fiber 170 having an input end 169 and an output end 171, and a beam shaping optical element 180. The beam shaping optical element 180 may be attached to the output end 171 of the illumination optical fiber 170. Although a plurality of the detection optical fibers 168 is illustrated in FIGS. 5 and 6, in other embodiments, at least one detection optical fiber 168 may be used. The distal tip 160 includes a scanner 185, which may be a MEMS scanner, mounted to interior of the housing 161. The illumination optical fiber 170 may be, for example, a single mode optical fiber. In some embodiments, the beam shaping optical element 180 may be a lens, refractive optical element, diffractive optical element, reflective optical element, or combinations thereof. A dome 164 may be affixed in a suitable manner to the housing 161 for sealing and protecting the components of the distal tip 160.

In various embodiment, the scanner 185 may be a 2D MEMS scanner, such as a bulk micro-machined MEMS scanner, a surface micro-machined device, another type of conventional MEMS scanner assembly, or a subsequently developed MEMS scanner assembly. The scanner 185 may be configured to scan one or more beams of light at high speed and in a pattern that covers an entire FOV or a selected portion of a 2D FOV within a frame period. As known in the art, such MEMS scanners may be driven magnetically, electrostatically, capacitively, or combinations thereof. For example, the horizontal scan motion may be driven electrostatically and the vertical scan motion may be driven magnetically. Electrostatic driving may include electrostatic plates, comb drives or the like. Alternatively, both the horizontal and vertical scan may be driven magnetically or capacitively.

FIG. 6 most clearly shows one embodiment for the scanner 185. The scanner 185 includes a scan plate 174 having a reflective surface 175, such as a polished surface or a suitable optical coating. The scan plate 174 is attached to a gimbal ring 172 by torsion arms 188 so that it may rotate about an axis 190 extending through the torsion arms 188. The gimbal ring 172 may also be attached to a frame 163 by torsion arms 186 so that it may rotate about an axis 192 extending through the torsion arms 186. Although not shown, it should be understood that the scanner 185 may include drive components common to MEMS scanners, such as drive circuitry and actuation components, for effecting rotation of the scan plate 174 about the axes 190 and 192. The scan plate 174 may also includes an aperture 178 extending through its thickness that is generally aligned with the output end 171 of the illumination optical fiber 170 to receive a beam of light shaped to a selected beam diameter by the beam shaping optical element 180 that can pass through the aperture 178.

The scanner 185 may include a plurality of openings formed therein. Openings 182a and 182b are defined by the gimbal ring 172, and the scan plate 174 and its associated torsion arms 188. Openings 184a and 184b are formed in the scanner 185 and are defined by the frame 163, and the gimbal ring 172 and its associated torsion arms 188. As best shown in FIG. 6, a collection end 173 of each of the detection fibers 168 are positioned aft of major plane P of the scanner 185 to receive light reflected from the FOV that passes through the openings 182a-182b and 184a-184b. Thus, preexisting openings in the scanner 185 may be used to receive reflected light from the FOV to enable making the distal tip 160 more compact.

The dome 164 may include a partially reflective interior reflective surface 176 for redirecting light emitted from the illumination optical fiber 170 to the scanner 185 and allowing light scanned from the scanner 185 to pass therethrough. In some embodiments, the dome 164 may be configured to provide optical power for shaping light it reflects to the scanner 185 and light scanned from the scanner 185 that passes through the dome 164. One embodiment of a suitable dome 164 is disclosed in the aforementioned '540 application. Such a dome is configured to selectively reflect and transmit light having a particular polarization direction. In other embodiments, the dome 164 may not have any optical power and a fixed intermediate reflective structure may be disposed between the surface 176 and the scanner 185.

In operation, light may be input into the input end 169 of the illumination optical fiber 170 using a light source (not shown) and emitted from the output end 171 of the illumination optical fiber 170 as beam 194. The beam 194 may be received by the beam shaping optical element 180, which is configured to focus the beam 194 to a selected shaped beam 196 that has a beam diameter smaller than the diameter of the aperture 178 through which it passes. After shaping and passing through the aperture 178 in the scan plate 174, the shaped beam 196 is reflected from an interior reflective surface 176 of the dome 164 to the reflective surface 175 of the scanner 185. As previously discussed above, the dome 164 may be configured to partially or fully collimate the shaped beam 196. Then, the scanner 185 and its associated reflective surface 175 scans the shaped beam 196 as a scanned beam 200 across the FOV. As the scanned beam 200 passes through the dome 164, it may be further shaped to a selected beam shape such as a beam having a selected beam waist distance from a distal end 177 of the dome 164. The scanned beam 200 is reflected off of the interior of a body cavity in which the distal tip 160 is positioned in. The reflected light (e.g., specular reflected light and diffuse reflected light also referred to as scattered light) from the FOV passes through the dome 164 and is received by respective collection ends 173 of the detection optical fibers 168 that are selectively positioned to receive the reflected light through one or more openings 182a-182b and 184a-184b in the scanner 185. Optical signals representative of characteristics of the FOV may be further processed to define an image.

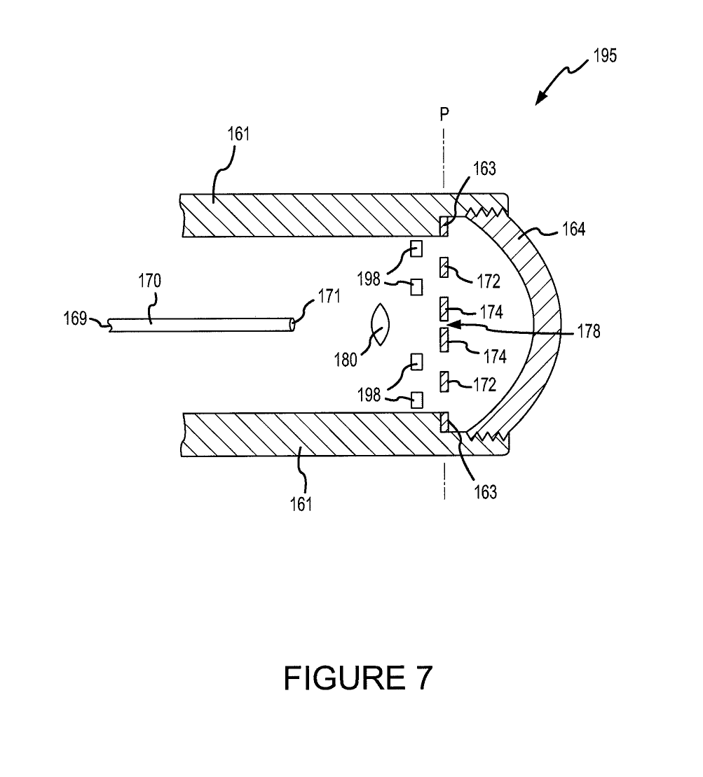

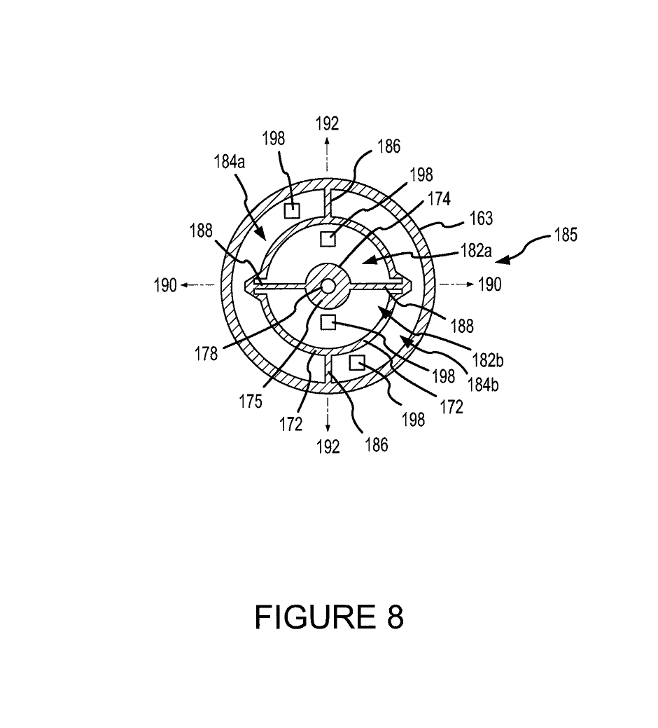

FIGS. 7 and 8 show a schematic partial side cross-sectional view and a front cross-sectional view of a distal tip 195, respectively, according to another embodiment. The distal tip 195 has many of the same components that are included in the distal tip 160 of FIGS. 4 through 6. Therefore, in the interest of brevity, the components of the distal tips 160 and 195 that correspond to each other have been provided with the same or similar reference numerals, and an explanation of their structure and operation will not be repeated. In the distal tip 195, light detection elements 198, such as PIN photodiodes or avalanche photodiodes, are used instead of the detection optical fibers 168. The light detection elements 198 may be selectively positioned to receive the reflected light from the FOV through the openings 182a-182b and 184a-184b. In the embodiment shown in FIGS. 7 and 8, four light detection elements 198 are employed, with each of the light detection elements 198 positioned aft of the major plane P behind a corresponding one of the openings 182a- 182b and 184a- 184b. In one embodiment, a first stage amplification such as a trans-impedance amplifier (TIA) may be integrated into the distal tip 195 to provide an amplified signal for transmission to the console of an endoscope. In another embodiment, two or more stages of amplification such as a TIA and AC-coupled voltage amplifier may be integrated into the distal tip 195 to provide even greater signal amplification. In yet another embodiment, an analog-to-digital (ADC) may be integrated into the distal tip 195 to provide a digitized signal for transmission to the console of the distal tip 195. In such an embodiment, a TIA first stage and AC-coupled voltage amplifier second stage, for example, may be used to improve signal-to-noise and reduce interference compared to analog transmission.

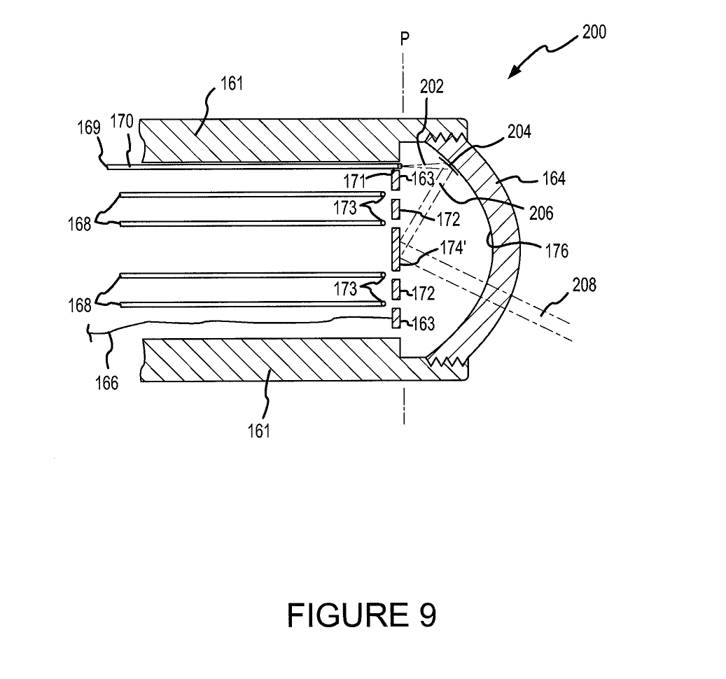

FIG. 9 shows a schematic partial side cross-sectional view of a distal tip 200 that may form part of or all of an endoscope tip according to another embodiment in which the illumination optical fiber 170 transmits light from a location laterally positioned in relation to the scanner 185 so that the light does not pass through an opening in the scanner 185. Such an embodiment may enable reducing the complexity of the optics because, for example, the beam shaping optical element 180 may be eliminated because the beam diameter does not have to be reduced to pass through an aperture formed in the scan plate of the scanner. Additionally, the size of the scan plate 174 of the scanner may be reduced because the aperture formed therein is eliminated. Accordingly, this may improve the performance characteristics of the scanner.

The distal tip 200 has many of the same components that are included in the distal tip 160 of FIGS. 5 and 6. Therefore, in the interest of brevity, the components of the distal tips 160 and 200 that correspond to each other have been provided with the same or similar reference numerals, and an explanation of their structure and operation will not be repeated. The output end 171 of the illumination optical fiber 170 of the distal tip 200 is laterally positioned in relation to the scanner 185 so that a beam 202 emitted therefrom does not pass through the scanner 185. Instead, the beam 202 may pass along the periphery of the scanner 185, and may be reflected from an intermediate reflective surface 204 that may partially or fully collimate the beam 202. Although the output end 171 of the illumination optical fiber 170 is shown laterally adjacent and generally coplanar with the scanner 185, in other embodiments, the output end 171 may be positioned forward or aft of the major plane P of the scanner 185 a selected axial distance. Reflected beam 206 from the intermediate reflective surface 204 is directed to the scan plate 174′ of the scanner 185, which scans it as scanned beam 204 across the FOV in the same manner as the distal tip 160. In another embodiment, the dome 164 may be used instead of the intermediate reflective surface 204 for reflection, collimation, or both of the beam 202. Of course, additional beam shaping optical elements, such as the beam shaping optical element 180, may be used, if desired, to control the beam shape and size output from the illumination optical fiber 170.

As with the distal tip 160, in the embodiment of the distal tip 200 shown in FIG. 9, the detection optical fibers 168 may also be positioned aft of the scanner 185 to receive the reflected light from the body cavity through one or more of the openings 182a-b and 184a-b in the scanner 185. However, in other embodiments, the detection optical fibers 168 may be positioned elsewhere, such as peripherally disposed about the housing 161. In yet a further embodiment, light detection elements 198 may be used instead of the detection optical fibers 168 as employed in the distal tip 195 of FIGS. 7 and 8.

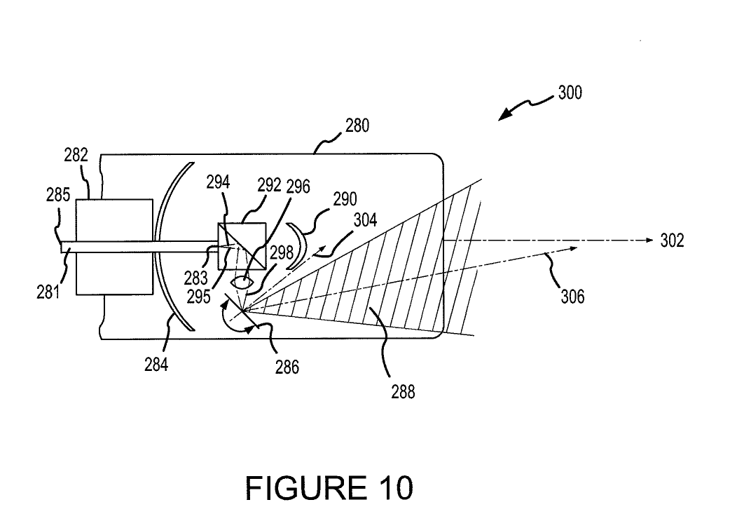

FIG. 10 shows a distal tip 300 configured to scan a beam across a non-axial FOV according to yet another embodiment. Thus, the distal tip 300 is a side-looking viewing device configured to provide a diagonal or side FOV. The distal tip 300 includes a housing 280 that encloses at least a portion of an optical fiber 281 having an input end 285 and an output end 283, a beam splitter 292, a large collection mirror 284, a scanner 286, and an optical element 290. The distal tip 300 has a longitudinal axis 302 that is non-parallel with a central normal axis 304 of the scanner 286 (the axis 304 is perpendicular to the scan plate of the scanner 286). By orienting the scanner 286 so that the axis 304 is non-parallel to the axis 302, the distal tip 300 has a non-axial FOV. Thus, the distal tip 300 can image a FOV that is off-axis relative to the longitudinal axis 302 of the distal tip 300. Although the scanner 286 is shown positioned to the side of the output end 285 of the optical fiber 281, in another embodiment, the scanner 286 may be positioned in front of the output end 285 and the light transmitted through an opening therein in a manner similar to the distal tip 160.

In operation, the optical fiber 281 outputs a beam 294 from the output end 285 and a portion of the beam 294 is redirected by the beam splitter 292 as redirected beam 295 to a collimation optical element 296. The collimation optical element 296, which may be one or more lenses, collimates or partially collimates the redirected beam 295 shown as beam 298. The scanner 286, which may be any of the aforementioned scanner configurations, scans the beam 298 as a scanned beam that is transmitted through the housing 280 or a window therein across a FOV 288. A central axis 306 of the FOV 288 is oriented at a non-zero angle relative to the longitudinal axis 302. Light reflected from the FOV is transmitted through the housing 280 and collected by the collection mirror 284. The light collected by the collection mirror 284 is reflected to the beam splitter 292, which transmits a portion of the light reflected from the collection mirror 284 to the optical element 290. The optical element 290 may be a curved mirror that focuses the light received from the beam splitter 292 and directs the light received from the beam splitter 292 back therethrough to the output end 285 of the optical fiber 281 for collection and transmission to an optical-electrical converter. Thus, in such an embodiment, additional detection optical fibers are not necessary because the optical fiber 281 acts as both an illumination optical fiber and a detection optical fiber.

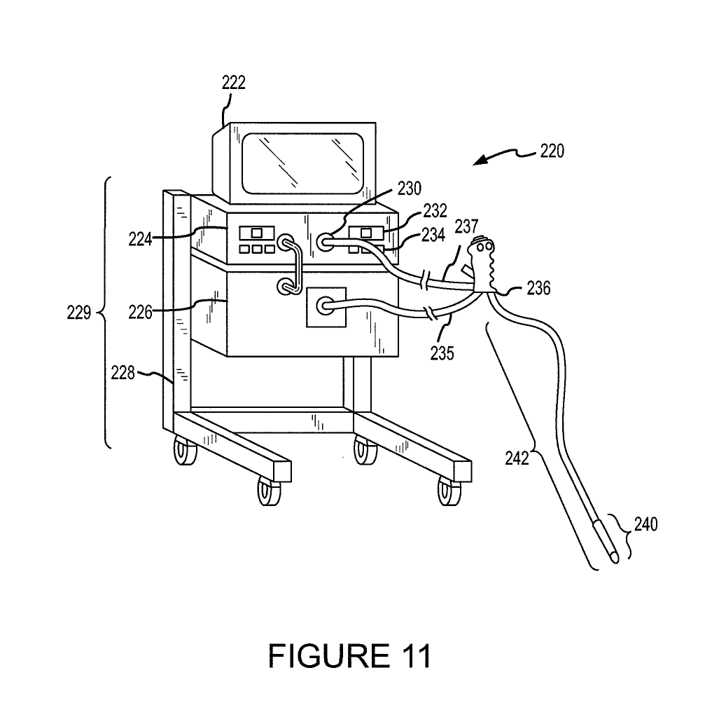

FIG. 11 shows a schematic drawing of a scanned beam endoscope 220 according to one embodiment that may utilize any of the aforementioned embodiments of distal tips. The scanned beam endoscope 220 includes a control module 224, monitor 222, and optional pump 226, all of which may be mounted on a cart 228, and collectively referred to as console 229. The console 229 may communicate with a handpiece 236 through an external cable 237, which is connected to the console 229 via connector 230. The handpiece 236 may be operably coupled to the pump 226 and an endoscope tip 242. The handpiece 236 controls the pump 226 in order to selectively pump irrigation fluid through a hose 235 and out of an opening of the endoscope tip 242. The endoscope tip 242 includes a distal tip 240, which may be any of the aforementioned embodiments of distal tips The endoscope tip 242 may encloses components of the distal tip 240, such as optical fibers and electrical wiring, and, optionally, other components such as an irrigation channel, a working channel, and a steering mechanism.

In operation, according to one embodiment, the distal tip 240 is placed within a body cavity. Responsive to user input via the handpiece 236, the distal tip 240 scans light over the FOV. Reflected light from the interior of the body cavity is collected by the distal tip 240. A photonic or electrical signal representative of an image of the internal surfaces is sent from the distal tip 240 to the console 229 for viewing on the monitor 222 and diagnosis by the medical professional. According to some embodiments, detection optical fibers, such as those shown in the embodiment of FIG. 5, transmit light to the console 229 for conversion to one or more electrical signals therein. According to other embodiments, one or wavelengths of light may be converted to corresponding electrical signals at the distal tip 240 using photodiodes as employed in the embodiment shown in FIG. 7. When photodiodes are employed, the corresponding electrical signals are transmitted to the console 229 for further processing.

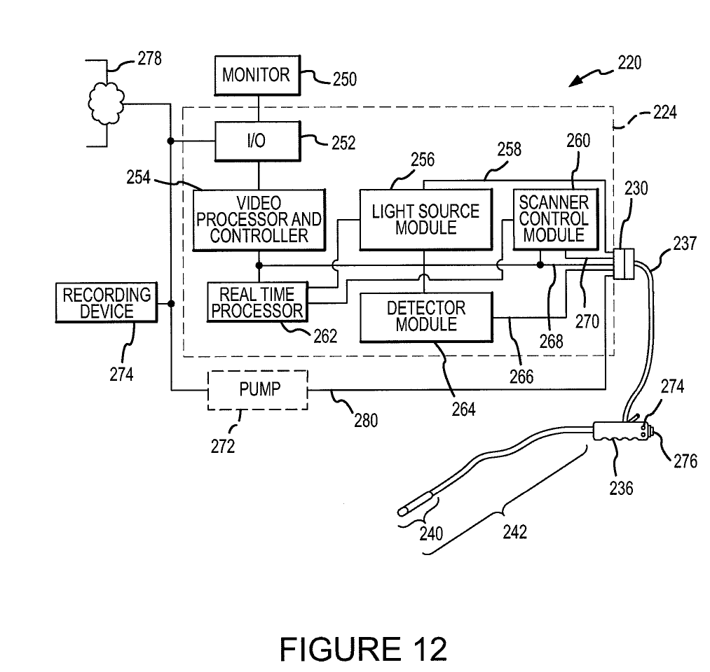

FIG. 12 is a block diagram illustrating the relationships between various components of the endoscope 220. The control module 224 contains a number of logical and/or physical elements that cooperate to produce an image on the monitor 222. The control module 224 includes a video processor and controller 254 that receives and is responsive to control inputs by the user via the handpiece 236. The video processor and controller 254 may also include image processing functions. The user control inputs are sent to the video processor and controller 254 via a control line 268.

The video processor and controller 254 also controls the operation of the other components within the control module 224. The control module 224 further includes a real time processor 262, which may, for example, be embodied as a PCI board mounted on the video processor and controller 254. The real time processor 262 is coupled to a light source module 256, a scanner control module 260, a detector module 264, and the video processor and controller 254. The scanner control module 260 is operable to control the scanner of the distal tip 240 and the detector module 264 is configured for detecting light reflected from the FOV.

The light source module 256, which may be housed separately, includes one or more light sources that provides the light energy used for beam scanning by the distal tip 240. Suitable light sources for producing polarized and/or non-polarized light include light emitting diodes, laser diodes, and diode-pumped solid state (DPSS) lasers. Such light sources may also be operable to emit light over a range of wavelengths.

Responsive to user inputs via the handpiece 236, a control signal is sent to the video processor and controller 254 via the control line 268. The video processor and controller 254 transmits instructions to the real time processor 262. Responsive to instructions from the real time processor 262, light energy is output from the light source module 256 to the endoscope tip 240 via an optical fiber 258. The optical fiber 258, which is optically coupled to the external cable 237 via the connector 230, transmits the light to the external cable 237. The light travels through the handpiece 236 to the distal tip 240 and is ultimately scanned across the FOV. Light reflected from the FOV is collected at the distal tip 240 and a representative signal is transmitted to the control module 224 using detection optical fibers or one or wavelengths of the reflected light may be converted to electrical signals and transmitted to the control module 224 using electrical wires.

In some embodiments, the representative signal transmitted to the control module 224 is an optical signal. Thus, a return signal line 266 may be an optical fiber or an optical fiber bundle that is coupled to the detector module 264 and transmit the representative optical signal to the detector module 264. At the detector module 264, the optical signals corresponding to the FOV characteristics are converted into electrical signals and returned to the real time processor 262 for real time processing and parsing to the video processor and controller 254. Electrical signals representative of the optical signals may be amplified and optionally digitized by the detector module 264 prior to transmission to real time processor 262. In an alternative embodiment, analog signals may be passed to the real time processor 262 and analog-to-digital conversion performed there. It is also contemplated that the detector module 264 and the real time processor 262 may be combined into a single physical element.

In other embodiments, reflected light representative of the FOV may be converted into electrical signals at the distal tip 240 or endoscope tip 242 by one or more photo-detectors such as PIN photodiodes, avalanche photodiodes (APDs), or photomultiplier tubes. In such an embodiment, the return line 266 may be electrical wires and the detector module 264 may be omitted. FIG. 7 shows a distal tip 195 of an endoscope tip that may be used in such an embodiment.

Continuing with the description of the block diagram of the endoscope 220, the video processor and controller 254 has an interface 252 that may include several separate input/output lines. A video output may be coupled to the monitor 222 for displaying the image. A recording device 274 may also be coupled to the interface 252 to capture video information recording a procedure. Additionally, in some embodiments, the endoscope system 220 may be connected to a network or the Internet 278 for remote expert input, remote viewing, archiving, library retrieval, or the like. In another embodiment, the video processor and controller 254 may optionally combine data received via the interface 252 with image data and the monitor 222 with information derived from a plurality of sources including the distal tip 240.

In another embodiment, in addition to or as an alternative to the monitor 222, the image may be output to one or more remote devices such as, for example, a head mounted display. In such an embodiment, context information such as viewing perspective may be combined with FOV and/or other information in the video processor and controller 254 to create context-sensitive information display.

From the foregoing it will be appreciated that, although specific embodiments of the invention have been described herein for purposes of illustration, various modifications may be made without deviating from the spirit and scope of the invention. For example, the teachings disclosed herein are generally applicable for use in scanned beam imagers, and bar code scanners in addition to scanned beam endoscopes. Accordingly, the invention is not limited except as by the appended claims.

What is claimed is:

1. A scanned beam endoscope, comprising:
a light source operable to provide light;
an endoscope tip, comprising:
an illumination optical fiber having an output end and an input end coupled to the light source;
a scanner positioned to receive a beam output from the output end of the illumination optical fiber and operable to scan the beam across a field-of-view (FOV), the scanner having a plurality of openings extending therethrough; and
at least one light detection element positioned to receive reflected light from the FOV through at least one of the openings in the scanner; and
a display coupled to the at least one light detection element, the display operable to show an image characteristic of the FOV.

2. The scanned beam endoscope of claim 1 wherein the at least one light detection element comprises a plurality of photodiodes positioned to receive the reflected light from the FOV through the plurality of openings in the scanner.

3. The scanned beam endoscope of claim 1:
wherein the at least one light detection element comprises at least one detection optical fiber positioned to receive the reflected light from the FOV through at least one of the openings in the scanner and transmit optical signals characteristic of the FOV;
further comprising a converter operable to convert the optical signals to electrical signals; and
wherein the display is coupled to the converter to receive the electrical signals.

4. The scanned beam endoscope of claim 1 wherein the at least one light detection element comprises at least detection optical fiber having a collection end positioned aft of a major plane of the scanner and oriented to receive the reflected light from the FOV.

5. The scanned beam endoscope of claim 1 wherein the at least one light detection element comprises a plurality of detection optical fibers.

6. The scanned beam endoscope of claim 5 wherein:
the scanner defines the plurality of openings; and
each of the detection optical fibers is positioned to receive the reflected light from the FOV through at least one of the openings.

7. The scanned beam endoscope of claim 1:
wherein the scanner comprises:
a frame;
a gimbal attached to the frame by first and second gimbal torsion arms, first and second openings of the plurality of openings being defined by the frame, gimbal, and first and second gimbal torsion arms; and
a scan plate having a reflective surface, the scan plate attached to the gimbal by first and second scan plate torsion arms, third and fourth openings of the plurality of openings defined by the gimbal, scan plate and first and second scan plate torsion arms; and
wherein the at least one light detection element comprises a plurality of detection optical fibers, each of the plurality of detection optical fibers being positioned to receive the reflected light from the FOV through at least one of the first, second, third, and fourth openings.

8. The scanned beam endoscope of claim 1 wherein the at least one light detection element is positioned aft of a major plane of the scanner.

9. The scanned beam endoscope of claim 1 wherein the scanner comprises an aperture and the illumination optical fiber is aligned with the aperture so that the beam output from the output end of the illumination optical fiber passes through the aperture.

10. The scanned beam endoscope of claim 9 wherein the endoscope tip comprises a beam shaping optical element positioned to receive the beam output from the output end of the illumination optical fiber, the beam shaping optical element operable to shape the beam to a selected beam size.

11. The scanned beam endoscope of claim 1 wherein the endoscope tip comprises a dome positioned to receive the beam scanned by the scanner and configured to shape the beam scanned by the scanner.

12. The scanned beam endoscope of claim 1 wherein the endoscope tip comprises a dome positioned to receive the beam scanned by the scanner, the dome being configured to reflect and transmit light having a particular polarization direction.

13. The scanned beam endoscope of claim 1 wherein the endoscope tip comprises a reflective surface positioned to redirect the beam to the scanner.

14. The scanned beam endoscope of claim 1 wherein the scanner comprises a MEMS scanner.

15. An endoscope tip, comprising:
an illumination optical fiber having an output end and an input end coupled to the light source;
a scanner positioned to receive a beam output from the output end of the illumination optical fiber and operable to scan the beam across a field-of-view (FOV), the scanner having a plurality of openings extending therethrough; and
at least one light detection element positioned to receive reflected light from the FOV through at least one of the openings in the scanner.

16. The endoscope tip of claim 15 wherein the at least one light detection element comprises a plurality of photodiodes positioned to receive the reflected light from the FOV through the plurality of openings in the scanner.

17. The endoscope tip of claim 15 wherein the at least one light detection element comprises at least one detection optical fiber positioned to receive the reflected light from the FOV through at least one of the openings in the scanner.

18. The endoscope tip of claim 15 wherein the at least one light detection element comprises at least one detection optical fiber having a collection end positioned aft of a major plane of the scanner and oriented to receive the reflected light from the FOV.

19. The endoscope tip of claim 15 wherein the at least one light detection element comprises a plurality of detection optical fibers.

20. The endoscope tip of claim 19 wherein:
the scanner defines the plurality of openings; and
each of the detection optical fibers is positioned to receive the reflected light from the FOV through at least one of the openings.

21. The endoscope tip of claim 15:
wherein the scanner comprises:
a frame;
a gimbal attached to the frame by first and second gimbal torsion arms, first and second openings of the plurality of openings being defined by the frame, gimbal, and first and second gimbal torsion arms; and
a scan plate having a reflective surface, the scan plate attached to the gimbal by first and second scan plate torsion arms, third and fourth openings of the plurality of openings defined by the gimbal, scan plate and first and second scan plate torsion arms; and
wherein the at least one light detection element comprises a plurality of detection optical fibers, each of the plurality of detection optical fibers being positioned to receive the reflected light from the FOV through at least one of the first, second, third, and fourth openings.

22. The endoscope tip of claim 15 wherein the at least one light detection element positioned aft of a major plane of the scanner.

23. The endoscope tip of claim 15 wherein the scanner comprises an aperture and the illumination optical fiber is aligned with the aperture so that the beam output from the output end of the illumination optical fiber passes through the aperture.

24. The endoscope tip of claim 23, further comprising a beam shaping optical element positioned to receive the beam output from the output end of the illumination optical fiber, the beam shaping optical element operable to shape the beam to a selected beam size.

25. The endoscope tip of claim 15, further comprising a dome positioned to receive the beam scanned by the scanner and configured to shape the beam scanned by the scanner.

26. The endoscope tip of claim 15, further comprising a dome positioned to receive the beam scanned by the scanner, the dome configured to reflect and transmit light having a particular polarization direction.

27. The endoscope tip of claim 15, further comprising a reflective surface positioned to redirect the beam to the scanner.

28. The endoscope tip of claim 15 wherein the scanner comprises a MEMS scanner.

29. A method of collecting light reflected from a field-of-view (FOV), comprising:
scanning a beam across the FOV using a scanner; and
transmitting at least a portion of light reflected from the FOV through at least one opening in the scanner for collection with at least one light detection element.

30. The method of claim 29 wherein the act of transmitting at least a portion of light reflected from the FOV through at least one opening in the scanner for collection with at least one light detection element comprises transmitting the at least a portion of the light reflected from the FOV through a plurality of openings defined by the scanner for collection with a plurality of detection optical fibers.

31. The method of claim 29, further comprising:
transmitting the beam through an aperture in the scanner; and
redirecting the beam transmitted through the aperture to the scanner.

32. The method of claim 29 wherein:
the at least one opening comprises a plurality of openings; and
the scanner comprises:
a frame;
a gimbal attached to the frame by first and second gimbal torsion arms, first and second openings of the plurality of openings being defined by the frame, gimbal, and first and second gimbal torsion arms; and
a scan plate having a reflective surface, the scan plate attached to the gimbal by first and second scan plate torsion arms, third and fourth openings of the plurality of openings defined by the gimbal, scan plate and first and second scan plate torsion arms; and
the act of transmitting at least a portion of light reflected from the FOV through at least one opening in the scanner for collection with at least one light detection element comprises transmitting the at least a portion of the light reflected from the FOV through the first, second, third, and fourth openings for collection with a plurality of detection optical element.

33. The method of claim 29 wherein the scanner is included in an endoscope tip of a scanned beam endoscope.

34. A scanned beam endoscope, comprising:
a light source operable to provide light;
an endoscope tip, comprising:
an illumination optical fiber having an output end and an input end coupled to the light source;
a scanner positioned to receive a beam output from the output end of the illumination optical fiber and operable to scan the beam across a field-of-view (FOV), the output end of the illumination optical fiber laterally positioned in relation to the scanner; and
at least one light detection element positioned to receive reflected light from the FOV; and
a display coupled to the at least one light detection element, the display operable to show an image characteristic of the FOV.

35. The scanned beam endoscope of claim 34 wherein the at least one light detection element comprises a plurality of photodiodes.

36. The scanned beam endoscope of claim 34:
wherein the at least one light detection element comprises at least one detection optical fiber positioned to receive the reflected light from the and transmit optical signals characteristic of the FOV;
further comprising a converter operable to convert the optical signals to electrical signals; and
wherein the display is coupled to the converter to receive the electrical signals.

37. The scanned beam endoscope of claim 34 wherein the output end of the illumination optical fiber is positioned laterally adjacent to a periphery of the scanner.

38. The scanned beam endoscope of claim 34 wherein the output end of the illumination optical fiber is positioned aft of a major plane of the scanner.

39. The scanned beam endoscope of claim 34 wherein the output end of the illumination optical fiber is positioned forward of a major plane of the scanner.

40. The scanned beam endoscope of claim 34 wherein:
the scanner defines a plurality of openings; and
the at least one light detection element comprises a plurality of detection optical fibers, each of the detection optical fibers is positioned to receive the reflected light from the FOV through at least one of the openings in the scanner.

41. The scanned beam endoscope of claim 34 wherein the endoscope tip comprises a beam shaping optical element positioned to receive the beam output from the output end of the illumination optical fiber, the beam shaping optical element operable to shape the beam to a selected beam size.

42. The scanned beam endoscope of claim 34 wherein the endoscope tip comprises a dome positioned to receive the beam scanned by the scanner and configured to shape the beam scanned by the scanner.

43. The scanned beam endoscope of claim 34 wherein the endoscope tip comprises a dome positioned to receive the beam scanned by the scanner, the dome configured to reflect and transmit light having a particular polarization direction.

44. The scanned beam endoscope of claim 34 wherein the endoscope tip comprises a reflecting surface configured and positioned to receive and shape the beam output from the output end of the illumination optical fiber.

45. The scanned beam endoscope of claim 34, further comprising a reflective surface positioned to redirect the beam to the scanner.

46. The scanned beam endoscope of claim 34 wherein the scanner comprises a MEMS scanner.

47. An endoscope tip, comprising:
an illumination optical fiber having an output end and an input end coupled to the light source;
a scanner positioned to receive a beam output from the output end of the illumination optical fiber and operable to scan the beam across a field-of-view (FOV), the output end of the illumination optical fiber laterally positioned in relation to the scanner; and
at least one light detection element positioned to receive reflected light from the FOV through at least one of the openings in the scanner.

48. The endoscope tip of claim 47 wherein the at least one light detection element comprises a plurality of photodiodes.

49. The scanned beam endoscope of claim 34 wherein the at least one light detection element comprises at least one detection optical fiber.

50. The endoscope tip of claim 47 wherein the output end of the illumination optical fiber is positioned laterally adjacent to a periphery of the scanner.

51. The endoscope tip of claim 47 wherein the output end of the illumination optical fiber is positioned aft of a major plane of the scanner.

52. The endoscope tip of claim 47 wherein the output end of the illumination optical fiber is positioned forward of a major plane of the scanner.

53. The endoscope tip of claim 47 wherein:
the scanner defines a plurality of openings; and
the at least one light detection element comprises a plurality of detection optical fibers, each of the detection optical fibers is positioned to receive the reflected light from the FOV through at least one of the openings in the scanner.

54. The endoscope tip of claim 47, further comprising a beam shaping optical element positioned to receive the beam output from the output end of the illumination optical fiber, the beam shaping optical element operable to shape the beam to a selected beam size.

55. The endoscope tip of claim 47, further comprising a dome positioned to receive the beam scanned by the scanner and configured to shape the beam scanned by the scanner.

56. The endoscope tip of claim 47, further comprising a dome positioned to receive the beam scanned by the scanner, the dome configured to reflect and transmit light having a particular polarization direction.

57. The endoscope tip of claim 47, further comprising a reflecting surface configured and positioned to receive and shape the beam output from the output end of the illumination optical fiber.

58. The endoscope tip of claim 47, further comprising a reflective surface positioned to redirect the beam to the scanner.

59. The endoscope tip of claim 47 wherein the scanner comprises a MEMS scanner.

60. A method of scanning light across a field-of-view (FOV), comprising:
transmitting a beam from a location lateral in relation to a scanner;
redirecting the beam to the scanner; and
scanning the redirected beam across the FOV.

61. The method of claim 60, further comprising collecting reflected light from the FOV with a plurality of light detection element.

62. The method of claim 61 wherein the plurality of light detection elements comprises a plurality of detection optical fibers.

63. The method of claim 61 wherein the plurality of light detection elements comprises a plurality of photodiodes.

64. The method of claim 60 wherein the act of transmitting beam from a location lateral in relation to a scanner comprises emitting the beam from an illumination optical fiber.

65. The method of claim 60 wherein the act of transmitting beam from a location lateral in relation to a scanner comprises emitting the beam from a position located laterally adjacent to a periphery of the scanner.

66. The method of claim 60 wherein the act of transmitting beam from a location lateral in relation to a scanner comprises emitting the beam from a location aft of the scanner.

67. The method of claim 60 wherein the act of transmitting beam from a location lateral in relation to a scanner comprises emitting the beam from a location forward of the scanner.

68. The method of claim 60, further comprising shaping the beam emitted from the illumination optical fiber.

69. The method of claim 60 wherein the act of redirecting the beam to the scanner comprises reflecting the beam off of a reflective surface.

70. The method of claim 60 wherein:
the scanner is included in an endoscope tip of a scanned beam endoscope; and
the act of redirecting the beam to the scanner comprises reflecting the beam off a dome of the endoscope tip.

71. The method of claim 60 wherein the scanner is included in an endoscope tip of a scanned beam endoscope.

72. A scanned beam endoscope, comprising:
a light source operable to provide light;
an endoscope tip, comprising:
an optical fiber having an output end and an input end coupled to the light source; and
a scanner positioned to receive a beam output from the output end of the optical fiber and operable to scan the beam across a field-of-view (FOV), a central normal axis of the scanner oriented at a non-zero angle relative to a longitudinal axis of the endoscope tip; and
a converter operable to covert optical signals characteristic of light reflected from the FOV to electrical signals; and
a display coupled to receive the electrical signals from the converter, the display being operable to show an image characteristic of the FOV.

73. The scanned beam endoscope of claim 72 wherein the scanner is positioned to one side of the longitudinal axis of the endoscope tip.

74. The scanned beam endoscope of claim 72 wherein the endoscope tip comprises:
a beam splitter positioned to receive the beam output from the output end of the optical fiber and configured to redirect the beam as a redirected beam to the scanner.

75. The scanned beam endoscope of claim 74 wherein the endoscope end comprises a collection mirror positioned to receive the reflected light from the FOV and redirect the reflected light to an optical element positioned and configured to focus the reflected light for collection by the optical fiber.

76. The scanned beam endoscope of claim 75 wherein the optical element comprises a curved mirror.

77. The scanned beam endoscope of claim 75 wherein the collection mirror comprises a curved mirror.

78. The scanned beam endoscope of claim 74 wherein the beam output from the optical fiber is transmitted through the scanner.

79. The scanned beam endoscope of claim 74 wherein optical fiber is configured to transmit the light reflected from the FOV as the optical signals to the converter.

80. An endoscope tip, comprising:
an optical fiber having an output end and an input end coupled to the light source; and
a scanner positioned to receive a beam output from the output end of the optical fiber and operable to scan the beam across a field-of-view (FOV), a central normal axis of the scanner oriented at a non-zero angle relative to a longitudinal axis of the endoscope tip.

81. The endoscope tip of claim 80 wherein the scanner is positioned to one side of the longitudinal axis of the endoscope tip.

82. The endoscope tip of claim 80, further comprising a beam splitter positioned to receive the beam output from the output end of the optical fiber and configured to redirect the beam as a redirected beam to the scanner.

83. The endoscope tip of claim 82, further comprising a collection mirror positioned to receive the reflected light from the FOV and redirect the reflected light to an optical element positioned and configured to focus the reflected light for collection by the optical fiber.

84. The endoscope tip of claim 83 wherein the optical element comprises a curved mirror.

85. The endoscope tip of claim 83 wherein the collection mirror comprises a curved mirror.

86. The endoscope tip of claim 80 wherein the beam output from the optical fiber is transmitted through the scanner.

87. The endoscope tip of claim 80 wherein optical fiber is configured to transmit the light reflected from the FOV.

88. A method of scanning a beam across a field-of-view (FOV) from an endoscope tip including a scanner, the method comprising:
scanning the beam across the FOV using the scanner, a central axis of the FOV oriented at a non-zero angle relative to a longitudinal axis of the endoscope tip.

89. The method of claim 88, further comprising:
collecting the light reflected from the FOV with the endoscope tip; and
redirecting the collected light to an optical fiber.

90. The method of claim 88 wherein the act of scanning the beam across the FOV using the scanner comprises scanning the beam across the FOV reflecting the beam from a scanner in which a central normal axis thereof is oriented at a non-zero angle relative to the longitudinal axis of the endoscope tip.
